(19) United States
(12) Plant Patent Application Publication
(10) Pub. No.: US 2004/0268455 P1
(43) Pub. Date: Dec. 30, 2004

(54) Chrysanthemum plant named 'Chamidi'

(75) Inventor: Jean-Pierre Challet, Nuaille (FR)

Correspondence Address: BURNS DOANE SWECKER & MATHIS L L P, POST OFFICE BOX 1404, ALEXANDRIA, VA, 22313-1404, US

(73) Assignee: SELECTION NEW PLANT SARL, Le Cannet Des Maures (FR)

(21) Appl. No.: 10/893,909

(22) Filed: Jul. 20, 2004

Related U.S. Application Data
(63) Continuation of application No. 10/114,446, filed on Apr. 3, 2002.

(51) Int. Cl. A01H 5/00

(52) U.S. Cl. PLT/284

(57) ABSTRACT

A new and distinct Chrysanthemum cultivar is provided that is a mutation of the ‘Chanizet’ variety (U.S. Plant patent application Ser. No. 10/114,447, filed concurrently herewith). Attractive ball-shaped bright yellow blossoms are formed in profusion. Such blossoms commonly are approximately 10 to 12 cm in diameter when fully open. The growth habit is low and compact and well-branched and yields a generally uniform and spherical overall plant. The foliage is glossy dark green and contrasts well with the bright yellow blossom coloration, and the stems are extremely stiff. The plant grows well in pots and is well suited for use as an attractive potted gift plant.

This is a Continuation of U.S. Plant patent application Ser. No. 10/114,446, filed Apr. 3, 2002.

### BOTANICAL/COMMERCIAL CLASSIFICATION

Chrysanthemum mortifolium/Decorative Pot Mum

### VARIETAL DENOMINATION

cv. ‘Chamidi’

### SUMMARY OF THE INVENTION

The present invention comprises a new and distinct cultivar of Chrysanthemum, botanically known as Chrysanthemum mortifolium, and hereafter is referred to by the cultivar name ‘Chamidi’.

The new cultivar of the present invention was created at Nuaillé, France when plants of the ‘Chanizet’ cultivar (U.S. Plant patent application Ser. No. 10/114,447, filed concurrently herewith) were irradiated with gamma radiation. The female parent of the ‘Chamidi’ variety was named ‘88/28/3’ (non-patented in the United States) and the male parent of the ‘Chamidi’ variety was named ‘87/172/4’ (non-patented in the United States). Cuttings of the irradiated plants were used to produce new plants that were carefully studied. The new cultivar of the present invention was selected from among the rooted cuttings at Nuaillé, France, primarily because of its distinctive and attractive flower coloration.

It was found that the new Chrysanthemum cultivar of the present invention displays:

(a) a low compact, well-branched, and generally spherical growth habit,

(b) forms attractive ball-shaped long-lasting decorative uniform bright yellow blossoms,

(c) forms extremely stiff stems,

(d) forms attractive dark green leaves having shiny upper surfaces, and

(e) an ability to grow well in pots to provide an attractive potted gift plant.

The new cultivar can be grown singly or in clumps in pots. It also can be grown in the landscape. The bright yellow blossoms contract nicely with the shiny dark green foliage. The blossoms have been found to be long lasting and commonly last up to four weeks on the plant. Pinching is helpful to further enhance branching; however, such pinching is not necessary since the plant already is naturally self-branching.

The new cultivar can be readily distinguished from its ‘Chanizet’ parent in view of the different blossom coloration. The blossoms of the cultivar of the present invention are a uniform bright yellow while those of the ‘Chanizet’ cultivar are bronze in coloration. Also, each of the ‘88/28/3’ and ‘87/172/4’ parental varieties of the ‘Chamidi’ cultivar display bronze-colored blossoms.

Asexual reproduction of the new cultivar by cuttings as performed at Nuaillé, France, in a controlled environment has demonstrated that the characteristics of the new cultivar are firmly fixed and are retained through successive generations of asexual propagation.

‘Chamidi’ has not been observed under all possible environmental conditions to date. Accordingly, it is possible that the phenotype may vary somewhat with variations in the environment, such as temperature, light, day length, contact with pesticides and/or subjection to grown regulation treatments.

### BRIEF DESCRIPTION OF THE PHOTOGRAPHS

The accompanying photographs depict important parts of the new ‘Chamidi’ cultivar when grown in a pot in a greenhouse at Nuaillé, France. The plant was approximately four months of age.

FIG. 1 shows a close view of an attractive ball-shaped bright yellow blossom of the ‘Chamidi’ cultivar.

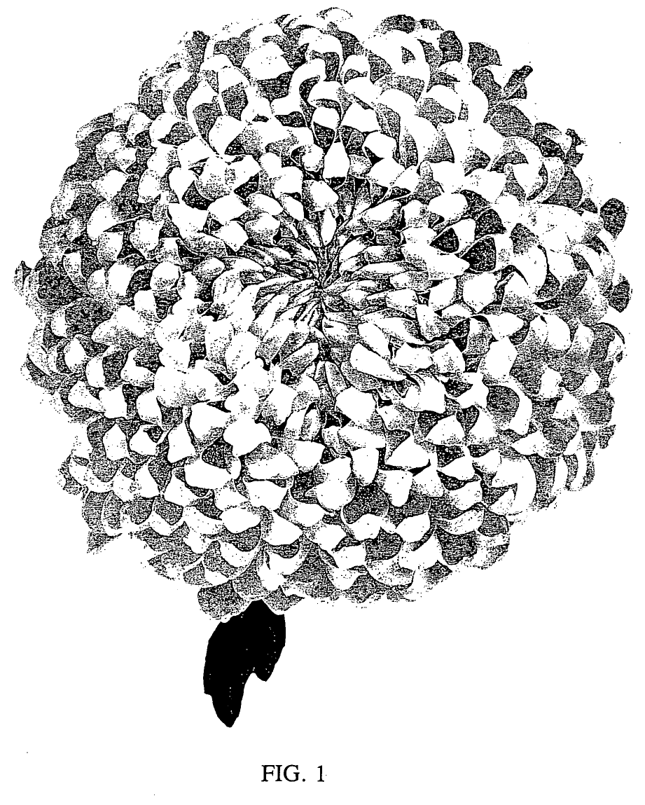

FIG. 2 shows a typical dark green leaf of the ‘Chamidi’ cultivar. While not readily apparent in a photograph of this character, the upper surface is glossy.

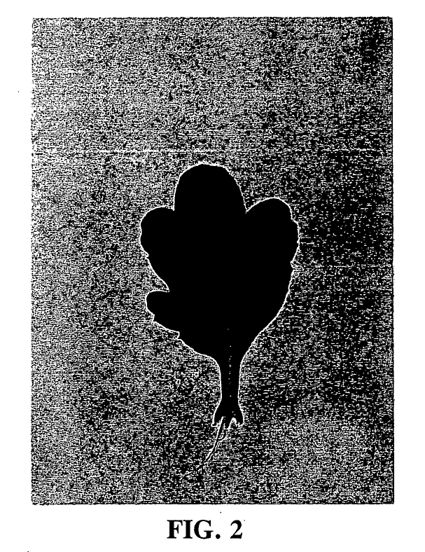

### DETAILED DESCRIPTION

The plants described were grown in pots in a greenhouse at Nuaillé, France. No growth regulation was used. The growing conditions approximate those commonly utilized for the commercial production of decorative pot mums.

General appearance: Short and stocky with a generally spherical growth habit.

Foliage: Attractive dark green. The upper and under surfaces of mature leaves are near Green Group 137A and 139A. The upper surfaces of the leaves also are shiny and tend to glisten in the sunlight. The leaf texture is smooth and fleshy. A typical leaf commonly displays an average length of approximately 7 to 10 cm and an average width of approximately 4 to 6 cm. The leaf apex is obtuse and the leaf base is somewhat attenuate. The petiole is near Green Group 137A and 139A in coloration. The main central venation is slightly lighter green (as illustrated). The leaf margin bears medium serration (as illustrated). The petiole commonly is approximately 1.5 to 2.5 cm in length on average, and commonly is approximately 0.5 to 0.8 cm in diameter on average.

Stems: Extremely stiff and smooth in texture. A typical flowering stem commonly is approximately 35 to 40 cm in length on average, and approximately 1.1 to 1.4 cm in diameter on average. The typical internode length commonly is approximately 2.5 to 3.5 cm on average.

Flowers: There commonly are approximately 6 to 8 flowers per stem. They possess a decorative uniform bright yellow coloration. There are no disc florets. The ray florets are concave in configuration and are near Yellow Group 7D on the outside and near Yellow Group 5A on the inside. The ray floret apex commonly is pointed. The ray florets commonly number at least 300 and are tubular at the base. The texture of the ray florets is somewhat roughened. The corolla tube is near Yellow Group 7D in coloration. The configuration is generally ball-shaped and the diameter is approximately 10 to 12 cm when fully expanded. A typical ray floret commonly displays an average length of approximately 7 to 13 cm and an average width of approximately 1 to 1.4 cm. The corolla tube commonly is approximately 6 to 11.5 cm in length on average. The buds are rounded at the tip, approximately 1.6 to 2.2 cm in length on average, approximately 2.5 to 3.5 cm in diameter on average, and near Yellow Group 7A in coloration. The peduncle is very strong, possesses a smooth texture, possesses a length of approximately 5 to 7 cm on average, possesses a diameter of approximately 0.8 to 1.2 cm on average, and commonly possesses a coloration of between Yellow-Green Group 144A and 146A. No reproductive organs or seeds have been observed to date.

Flower duration: Very long lasting with the blossoms commonly lasting up to approximately four weeks on the plant.

Response time: The time between the beginning of the short day period and the flowering date at normal temperature and light intensity is approximately ten weeks.

Hardiness: Has satisfactorily withstood temperatures in the range of 0 to 2° C.

Usage: Decorative pot mum. No noteworthy resistance or susceptibility to disease, insects, or drought has been encountered during observations to date.

I claim:

1. A new and distinct cultivar of Chrysanthemum plant that displays:
(a) a low compact, well-branched, and generally spherical growth habit,
(b) forms attractive long-lasting decorative ball-shaped uniform bright yellow blossoms,
(c) forms extremely stiff stems,
(d) forms attractive dark green leaves having shiny upper surfaces, and
(e) an ability to grow well in pots to provide an attractive potted gift plant;
substantially as illustrated and described.
